(19) United States
(12) Patent Application Publication
(10) Pub. No.: US 2010/0286257 A1
(43) Pub. Date: Nov. 11, 2010

(54) Methods Of Use Of Nitroalkane Compositions In Dermatologic Applications To Prevent or Treat Skin Aging

(76) Inventor: Nicholas V. Perricone, Meriden, CT (US)

Correspondence Address: ST. ONGE STEWARD JOHNSTON & REENS, LLC, 986 BEDFORD STREET, STAMFORD, CT, 06905-5619, US

(21) Appl. No.: 12/463,052

(22) Filed: May 8, 2009

(51) Int. Cl. A61K 8/40; A61K 8/49; A61Q 19/08

(52) U.S. Cl. 514/458; 514/560

(57) ABSTRACT

Topical compositions comprising an effective amount of a nitroalkene and a carrier are used to prevent or treat skin aging.

### FIELD OF THE INVENTION

The present invention relates to topical nitroalkene compositions to improve skin conditions. The nitroalkene compositions may be used to prevent skin damage, and to treat skin damage, particularly skin inflammation. Methods of use include treatment of rosacea, eczema, psoriasis, xerosis, dermatitis, seborrhea, thermal and radiation burns (including sunburn), acne, alopecia, skin aging, scars, and skin inflammation.

### BACKGROUND OF THE INVENTION

The metabolism of arachidonic acid is a key element of inflammation. In acute inflammation, there is typically a respiratory burst of neutrophil activity that initiates cascades involving a change in the oxidation state of the cell. Alteration in the redox state of the cell activates transcription factors such as NFκB as well as AP1, which then causes production of proinflammatory mediators. These mediators, such as Tumor necrosis factorA (TFα) and various interleukins, cause a burst of other cytokines. Arachadonic acid is released, which is oxidized to biologically active mediators. When arachadonic acid is oxidized via the cyclooxygenase or lipoxygenase pathways, eicosanoids e.g. prostaglandins, leukotrines, and hyroxyeicosatetraenoic acid (HETE) are produced, which cause erythma, edema, and free radical production.

Free fatty acids and esterified fatty acids are important components of lipoproteins and membranes. They react with nitric oxide (.NO) and nitric oxide derived species (NOx) to produce a variety of oxidized and nitrated products. The oxidation of polyunsaturated fatty acids plays an important role in biological systems and some of the metabolic products from polyunsaturated fatty acid oxidation are important biological mediators. The nitrated lipids produced act as signaling mediators leading to secondary changes in protein function via electrophilic based modifications. Nitric oxide and its metabolites also induce cyclic guanosine monophosphate (cGMP)-independent actions in host defense mechanisms and cell signaling. Moreover, the reaction of NOx with eicosanoids and their impact on biosynthetic enzymes are significant elements in the modulation of inflammatory response. Reactions of .NO and .NO metabolites can influence catalytic reactions in eicosanoid synthesis and modulated gene expression of related enzymes. Further, the transcription factor NFkB mediates inducible nitric oxide synthase expression in LPS-activated macrophages. And, —NO can serve to down-regulate initial lipoid-mediated signaling events. Nitrated fatty acids, particularly, nitroalkene derivatives of fatty acids, have been detected in vivo in the blood and urine of healthy humans. (Baker et al., J. Biol. Chem., 2005 280:42464-42475).

.NO is an endogenously generated, lipophilic signaling molecule that maintains vascular homeostasis via stimulation of soluble guanylate cyclase. In addition to mediating vascular relaxation, .NO potently modulates oxygen radical reactions, inflammatory cell function, post-translational protein modification and regulation of gene expression. There are multiple pathways whereby .NO-derived species can mediate the oxidation and nitration of biomolecules such as unsaturated fatty acids.

Acute inflammation is often characterized generation of excited oxygen species, e.g. superoxide anion, which damages the lipid-rich membranes and activate the chemical mediators of the proinflammation and inflammation cascades. These oxygenated species tend to concentrate in hydrophobic regions. Both in or near these hydrophobic compartments, .NO and NOx undergo a rich spectrum of reactions with oxygen species, transition metals, thiols, lipids, and a variety of organic radicals. These multifaceted reactions yield reactive species that transduce .NO signaling and modulate tissue inflammatory responses.

Nitric oxide reacts with superoxide (O₂⁻) to yield peroxynitrite (ONOO⁻) and its conjugate acid, peroxynitritrous acid (ONOOH), the latter of which undergoes homolytic scission to nitrogen dioxide (NO₂) and hydroxyl radical (OH). Also, ONOO⁻ can react with CO₂, to form nitrosoperoxycarbonate (ONOOCO₂⁻), which breaks down to NO² and carbonate (CO₃—) radicals via homolysis, or rearrangement to NO₃⁻ and CO₂.

During inflammation, adaptive and protective responses are elicited by vascular and other tissues to protect the host from its own mechanisms directed at destroying invading pathogens. Heme oxygenase 1 (HO-1) plays a central role in vascular inflammatory signaling and mediates a protective response to inflammatory stresses such as atherosclerosis, acute renal failure, vascular restenosis, transplant rejection, and sepsis Heme oxygenase 1 catalyzes the degradation of heme to biliverdin, iron, and CO, the last of which has been shown to display diverse, adaptive biological properties, including anti-inflammatory, anti-apoptotic, and vasodilatory actions. During inflammation, HO-1 gene expression is up-regulated, with induction typically occurring transcriptionally. Neutrophil myeloperoxidase and heme proteins such as myoglobin and cytochrome c catalyze H₂O₂-dependent oxidation of nitrite (NO₂⁻) to NO₂, resulting in biomolecule oxidation and nitration that is influenced by the spatial distribution of catalytic heme proteins. These and other products are capable of concerted oxidation, nitrosation and nitration of target molecules.

The body contains an endogenous antioxidant defense system made up of antioxidants such as vitamins C and E, glutathione, and enzymes, e.g., superoxide dismutase. When metabolism increases or the body is subjected to other stress such as infection, extreme exercise, radiation (ionizing and non-ionizing), or chemicals, the endogenous antioxidant systems are overwhelmed, and free radical damage takes place. Over the years, the cell membrane continually receives damage from reactive oxygen species and other free radicals, resulting in cross-linkage or cleavage or proteins and lipoproteins, and oxidation of membrane lipids and lipoproteins. Damage to the cell membrane can result in myriad changes including loss of cell permeability, increased intercellular ionic concentration, and decreased cellular capacity to excrete or detoxify waste products. As the intercellular ionic concentration of potassium increases, colloid density increases and m-RNA and protein synthesis are hampered, resulting in decreased cellular repair. Some cells become so dehydrated they cannot function at all.

It would be desirable to have topical treatments for rosacea, eczema, acne, alopecia, psoriasis and inflammatory conditions in general using compositions which disrupt the inflammatory cascades describes above.

### SUMMARY OF THE INVENTION

The present invention is directed at the selection, formulation, and use of compounds which act with a protective response to prevent and attenuate inflammation to provide a therapeutic effect in their control of the pathological inflammation processes, and are also important in providing useful biochemical tools for mechanistic investigation of the enzymes involved.

Lipid nitration provides a means by which the proinflammatory aspects of reactive oxygen and nitrogen species and eicosanoids are down-regulated. The present invention is directed at the topical use of nitroalkene compositions, including particularly, nitrolinoleic acid, nitrooleic acid, nitrated species of arachidonic acid and nitrated cholesteryl lineolate, as lipid signaling mediators to reduce inflammation and inflammation mediated skin conditions.

It is an object of the invention to provide therapeutically effective topical compositions of nitroalkene and carrier to prevent, treat, or otherwise improve the skin conditions through topical application.

It is an object of the invention to provide methods for preventing and/or treating skin damage that comprise applying a composition containing nitroalkene in a dermatologically acceptable carrier to skin.

In accordance with the present invention, topical methods of use of nitroalkenes to prevent or treat rosacea, eczema, psoriasis, xerosis, dermatitis, seborrhea, acne, alopecia, other types of skin inflammation, skin aging, and scarring are disclosed.

The amount of nitroalkene necessary to treat skin or prevent skin damage is not fixed per se and is necessarily dependent upon the amount and identity of any adjunct ingredients in the preparation. In some typical embodiments of the invention, the composition comprises about 0.025% to about 70% by weight nitroalkene in a dermatologically acceptable polymer polyether and/or phosphatidycholine carrier. Optionally, at least one or a mixture of lipoic acid, fatty acid ester of ascorbic acid may be added to the composition.

In some typical embodiments of the invention, the method for preventing and/or treating skin damage comprises applying a composition containing about 0.025% to about 70% by weight of nitroalkene in a dermatologically acceptable carrier. Optionally, at least one or a mixture of lipoic acid or fatty acid ester of ascorbic acid may be added to the composition.

### DETAILED DESCRIPTION OF THE INVENTION

U.S. Pat. No. 6,924,309 to Ferrante et al., suggests that hydroxyl, hydroperoxy, epoxy and peroxy substituted nitro compounds may be useful due to their ability to inhibit IFN-γ. Such compounds are alkenes, typically containing 3 or more double bonds which must be formed through synthetic reactions such as the Michael addition.

Nitrated fatty acids serve as mediators of physiological and pathophysiological cell signaling processes. Functional consequences of these signaling mechanisms have been shown in inhibition of platelet and neutrophil functions, activation of the transcription factor Nrf2 which upregulates gene expressions of cytoprotective phase 2 protienases such as heme oxygenase-1 (HO-1), inhibition of LPS-induced cytokine release in moncytes, increased insulin sensitivity and glucose uptake in adipocytes, and relaxation of preconstricted rat aortic segments.

The mechanism of .NO release by nitrated fatty acids is not fully understood. Modified Nef reaction mechanisms (Schopfer et al., J. Biol. Chem. 2005 280:19289-97) and isomerization of the nitro-fatty acid to the corresponding nitrite derivative through a hemolytic scission of the —NO₂ group have been proposed (Lima et al., Free Radic. Biol. Med. 2005 39:532-39). Another proposal is that upon administration in vivo, nitro fatty acids undergo reversible and exchangeable electrophilic reactions with nucleophilic targets and are metabolized predominantly via saturation of the double bond and beta-oxidation reactions that terminate at the site of acyl-chain nitration (Rudolph et al., J. Biol Chem, 2009 284:1461-73). Reversible nitroalkylation reactions with glutathione (GSH) and the Cys and H is residues of proteins, demonstrate the electrophilic nature of the β-carbon adjacent to the nitro-bonded carbon. Nitrated fatty acids have been reported as potential endogenous ligands for PPARγ because of their ability to react with cellular nucleophiles to postranslationally modify protein structure, function, and localization (Baker et al., Free Radic Biol Med, 2009 46:989-1003).

Nitro-oleic acid (OANO₂) has been reported to inhibit xanthine oxidoreductase (XOR) activity, which generates proinflammatory oxidants and secondary nitrating species, with an IC50 of 0.6 μM (Kelley et al., J Biol Chem, 2008 283:36176-84). Glyceraldehyde-3-phosphate dehydrogenase (GAPDH) inhibition by nitro-oleic and -linoleic acid displayed an IC₅₀ of 3 μM, indicating a pathway for redox regulation of enzyme function, cell signaling and protein trafficking (Batthyany et al., J Biol Chem, 2006 281:20450-63). Structure function studies of nitro-oleic acid indicate that the carboxylic acid moiety, nitration at the 9 or 10 olefinic carbon, and unsaturation is required for XOR inhibition. OANO₂ has been reported to activate transcription factor Nrf2 to upregulate gene expression of HO-1 and other phase 2 protienases.

In 2004, Baker et al. reported the isolation of two positional isomers of nitro-linoleic acid (LNO₂) that were found in red blood cell membranes and plasma (Proc. Natl. Acad. Sci. U.S.A. 2004 101:11577-82). Since then, LNO₂ has been shown to inhibit vascular smooth muscle cell proliferation by activating the nuclear factor-erythroid 2-related factor 2 (Nrf2) (Villacorta et al., Am J Physiol Heart Circ Physiol, 2007 293:H770-6). Nrf2 is a transcription factor that is in the inactive form at the cytosol due to the inhibitory activity of Keap1. Keap1 is highly reactive to nitroalkylation since it constitutes a cysteine-rich protein. When activated, Nrf2 migrates to the nucleus and binds as a heterodymer to the antioxidant response element (ARE) in DNA, activating the expression of cytoprotective phase 2 enzymes i.e. heme oxygenase 1 (HO-1), superoxide dismutase, catalase, glutathione peroxidases, the peroxy redoxines, NADPH, and quinone oxyreductases.

LNO₂ also inhibits fMLP and PMA-mediated active of human neutrophils and blocks NF-kB activity, inhibits Keap1, resulting in activation of Nrf2 which induces expression of cytoprotective molecules. Moreover, LNO₂ and OANO₂ have been shown to exert cell signaling action via ligation and activation of PPARγ. PPARγ activation can effect modulation of metabolic and cellular differentiation genes and regulation of inflammatory responses. In the vasculature, PPARγ is expressed in monocytes, macrophages, smooth muscle cells, and endothelium and plays a central role in regulating the genes related to lipid trafficking, cell proliferation and inflammatory signaling. The removal of LNO₂-GSH adducts by MRP1 shows that electrophilic reactivity likely plays a role in inhibiting LNO₂ dependent PPARγ transcription.

Recently, studies on the role of HO-1 were reported. HO-1 plays a central role in vascular inflammatory signaling reactions and mediates a protective response. LNO₂ was shown to induce pulmonary epithelial HO-1 mRNA expression and adaptive responses to inflammation via both transcriptional and translational regulatory mechanisms (Iles et al., Free Radical Biology & Medicine 46 (2009) 866-75). Wright reported that LNO₂ mediates the induction of HO-1 by PPARγ independent and both .NO-dependent and independent mechanisms. Trotchansky and Rubbo confirmed the PPAR-γ independent mechanisms and reported that HO-1 induction by LNO₂ occurs predominantly by .NO-independent mechanisms (Free Radical Biology & Medicine 44 (2008) 1887-96).

The use of nitroalkenes in topical applications for improvement of skin conditions has not been described in the literature. The present invention comprises topical nitroalkene treatments which improve skin condition by disrupting the cascade of reactions that cause inflammation.

Nitroalkenes consist of the general formula NO₂-A-B, in which A is a saturated hydrocarbon chain and B is (CH₂)ₙ(COOH)ₘ in which n is 0 to 2 and m is 0 to 2; and the derivatives thereof having further one or more substitution selected from the group consisting of hydroxyl, hydroperoxy, epoxy and perxoy. In a preferred embodiment of the invention, A is a hydrocarbon chain of 17 atoms and B is CH₂(COOH). More specifically, the preferred compounds are nitro-linoleic acid, nitro-oleic acid, nitrated arachidonic acid, or nitrated cholesteryl lineolate. Of these, nitro-linoleic acid, and nitro-oleic acid are preferred.

Additional nitroalkene compounds that may be used in accordance with the invention include the compounds disclosed in Ferrante, U.S. Pat. No. 6,924,309; and Freeman, U.S. Patent Publication No. US 2007/0232579 A1, the disclosures of which are hereby incorporated by reference, those discussed by Trostchansky and Rubbo, Free Radical Biology & Medicine 44 (2008) 1887-96; and Baker et al., Free Radical Biology & Medicine, 46 (2009) 989-1003, the disclosures of which are incorporated herein by reference.

The most preferred compounds are those in which a NO₂ group is located adjacent a double bond in the carbon chain, such as in the compounds illustrated in Table 1 below.

TABLE 1

| Name | Formula | Structure |
|---|---|---|
| Nitrated oleic acid 9- and 10-nitro-cis- octedecenoic acids | OA- NO₂ | 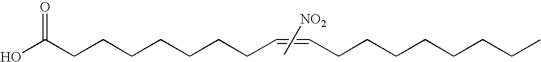 |
| Nitrated linoleic acid 9-, 10-, 12- and 13-nitro- cis-octedecadienoic acids | LNO₂ | 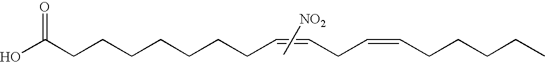 |
| | | 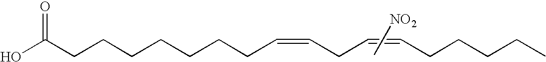 |
| | | 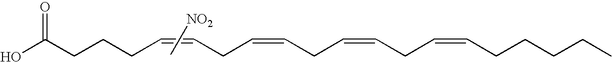 |
| Nitrated arachidonic acid 5-, 6-, 8-, 9-, 11-, 12-, 14- and 15-nitro-cis- elcosatetraenoic acids | AA- NO₂ | 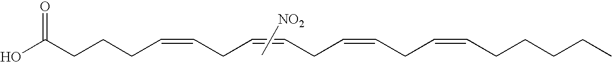 |
| | | 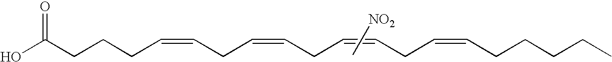 |
| | | 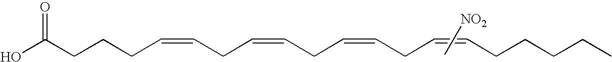 |
| Nitrated cholesteryl linoleate cholesteryl- 9-, 10-, 12- and 13-nitro-cis- octedecadienoates | CLNO₂ | 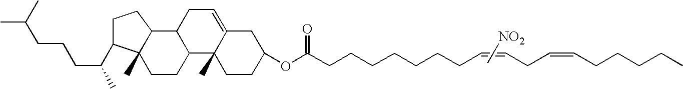 |
| | | 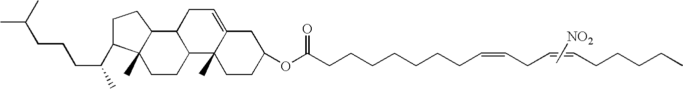 |

Polyunsaturated nitro compounds have been compared to fatty acids, which have a variety of biological activities including anti-inflammatory properties, since the nitro group is chemically similar to COOH groups of essential fatty acids with regard to size, charge and shape. In addition, the nitro compounds are a group of relatively stable compounds and are resistant to β-oxidation by preventing CoA thiosester production, which is the first step in β-oxidation of fatty acids. Because of this, they are not readily incorporated into lipids and are more likely to be present in a free form. Polyunsaturated nitro compounds have the ability to penetrate cells and tissues suggesting their use to prevent oxidative damage including anti-aging agents. Moreover, their ability to inhibit interferon-gamma (IFN-γ) (a cytokine of Th-1 cells) makes the substances useful in the treatment of allergy and skin diseases where IFN-γ plays a pathogenic role e.g. atopic dermatitis.

Synthesis and Formation

There are various experimental approaches to chemically synthesize nitrated unsaturated fatty acids. Preparation of nitroalkenes of the present invention may be possible through any of the routes disclosed in U.S. Pat. No. 6,924,309, and in Trostchansky and Rubbo supra.

Lipid nitration in vivo may also arise through one or more of several different pathways, namely: 1) nitrogen dioxide radical reacts with unsaturated lipids and lipid radicals leading to isomerized, oxidized and/or nitro-allylic, nitroalkane, dinitro, or nitro-hydroxy lipid derivatives; 2) peroxynitrite and peroxynitrous acid homolyze yielding nitrogen dioxide radical and hydroxyl radical which mediate oxidation, nitrosation, and nitration reactions; 3) addition of nitronium ion by electrophilic substitution at the double bond; 4) reaction of a carbon-centered radical with nitrogen dioxide radical both coming from a caged radical rearrangement of unstable alkyl peroxynitrite intermediates; and 5) nitroaldol addition by combining known precursors yielding a nitro-alcohol product i.e. activation of hydroxyl group followed by dehydration reaction via catalytic base.

Compositions

Only effective amounts of topical compositions containing nitroalkene are needed to achieve the intended benefits including prevention and treatment of inflammatory skin conditions, aging and scarring. By “effective amount” is meant an amount of active ingredient(s) sufficient to turn on the Nrf2 transcription factor and inhibit NF-kB and/or upregulate expression of other protective ligands, thereby inhibiting the products of the arachidonic acid cascade which leads to the activation of transcription factors that direct the cell nucleus in producing pro-inflammatory cytokines.

The topical compositions are based on a carrier in which the nitroalkene is soluble per se or is effectively solublized (e.g. as an emulsion or microemulsion). The carrier is dermatologically acceptable in the sense of not bringing about any adverse effect on the skin areas to which it is applied. The carrier preferably is appropriately selected for topical application, and forms a film or layer on the skin to which it is applied so as to localize the application. The nitroalkene is applied in admixture with the dermatologically acceptable carrier or vehicle (e.g. as a lotion, cream, gel, ointment, soap, stick, or the like) to as to facilitate topical application and provide therapeutic effects.

Non-polar and hydrophobic carriers are required for the compositions of the invention. Aqueous solvents and other polar solvents should be avoided because nitroalkenes are unstable in such solvents. Carriers may include polyethylene glycol, including PEG-1000, PEG-200, PEG-400; PEG-600; Labrasol® (a lipid-based self-emulsifying excipient mainly composed of PEG esters and glycerides with medium acyl chains); glycerin; polypropylene glycol; Stabileze® 06 (a PVM/MA Decadiene Crosspolymer); hydrogenated polyisobutane/polyethane; Permethyl® 99A (isododecane); BV-OSC (tetrahexyldecyl ascorbate); VC-IP (tetrahexyldecyl ascorbate); Vitamine E; beta carotene; disopropyl adipate; 2-ethylhexyl pentate; oleth-3; Ceraphyl® 31 (Propanoic acid 2-hydroxy-dodecyl ester); Ceraphyl® 41 (Propanoic acid, 2-hydroxy-, C12-15-alkyl esters); Glycereth-4; Glycereth-7; diglycerin; panthenol; and phytantriaol. Carrier formulations based principally on polymer polyethers such as polyethylene glycol and polypropylene glycol are a preferred embodiment.

A phosphatidycholine based carrier is another possible embodiment. Phosphatidylcholine, commonly called lecithin, is a mixture of diglycerides of stearic, palmitic, and oleic acids, linked to the choline ester of phosphoric acid. It can be isolated from eggs, soybeans, and other biological materials, chemically synthesized, or obtained commercially from many sources. Carrier formulations as disclosed in U.S. Pat. No. 7,182,956, the disclosure of which is hereby incorporated by reference, including polyenylphosphatidycholine enriched phosphatidycholine and polyglycol mixtures, are particularly preferred.

The quantity of the nitroalkene active ingredient in the carrier may be varied or adjusted widely depending upon the particular application, the potency of the particular compound, and the desired concentration. Generally, the quantity of nitroalkene active ingredient will range between 0.025% to 70% by weight of the topical composition. Generally, lower concentrations of nitroalkene active ingredients in a carrier are suitable, depending upon the application regimen and the active and adjunct ingredients employed.

The following weight percentage of nitrolalkene ranges are expected to be useful for different applications. These weight percentage ranges are applicable particularly to LNO₂ and OANO₂.

Weight Percentage

0.01%-0.025%

0.025%-0.05%

0.05%-0.10%

0.10%-0.50%

0.50%-1.0%

0.025%-0.50%

0.025%-1.0%

1.0%-2.0%

2.0%-5.0%

5.0%-10.00%

1.0%-5.0%

1.0%-10.0%

10.0%-20.0%

20.0%-30.0%

30.0%-40.0%

40.0%-50.0%

50.0%-60.0%

60.0%-70.0%

70.0%-80.0%

80.0%-90.0%

90.0%-98.0%

10.0%-30.0%

20.0%-40.0%

30.0%-60.0%

40.0%-70.0%

50.0%-80.0%

10.0%-50.0%

10.0%-98.0%

50.0%-70.0%

50.0%-98.0%

70.0%-98.0%

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

As nitroalkenes are very reactive molecules a nitroalkene topical composition desirably includes a substantial antioxidant and preservative system. In one preferred embodiment, the antioxidant system is Oxynex™ AP, Oynex™ LM, or Oxynex™ K. The preferred embodiments uses fatty acids of Vitamin C, specifically ascorbyl palmitate, as a significant component of the antioxidant system. Antioxidants are typically present in an amount ranging from about 0.025% to about 5.00% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. As mentioned above, particularly preferred antioxidants are those that provide additional benefits to the skin such as ascorbyl palmitate. Preservatives are typically present in an amount ranging from about 0.5% to about 2.0% by weight percent, based on the total composition.

Emollients, typically present in amounts ranging from about 0.01% to 5% of the total composition include, but are not limited to, fatty esters, fatty alcohols, mineral oils, polyether siloxane copolymers, and mixtures thereof. Humectants may be present in amounts ranging from about 0.1% to about 5% by weight of the total composition. Non-polar humectants are preferred. Emulsifiers, typically present in amounts from about 1% to about 10% by weight of the composition, include, but are not limited to, stearic acid, cetyl alcohol, stearyl alcohol, steareth 2, steareth 20, acrylates/C10-30 alkyl acrylate cross-polymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Some embodiments of this invention contain at least one other adjunct ingredient in addition to nitroalkene(s). Fat-soluble fatty acid esters of ascorbic acid (vitamin C) are employed as an adjunct ingredient as well as an antioxidant in some embodiments. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. Ascorbyl palmitate is used in one prefrerred embodiment. Other possible adjunct ingredients include, but are not limited to one or more of: amino acids, lipoic acid; or tocotrienols and tocotrienol derivatives and vitamin E compo-sitions enriched with tocotrienols or tocotrienol derivatives

Additional ingredients and methods disclosed in U.S. Pat. Nos. 4,775,530, 5,376,361, 5,409,693, 5,545,398, 5,574,063, 5,643,586, 5,709,868, 5,879,690, 5,965,618, 6,051,244, 6,162,419, and 6,191,121 to Perricone are hereby incorporated by reference.

Proposed example formulations are as follows.

### Example 1

A nitroalkene topical composition formulation is as follows.

| Component | % w/w |
|---|---|
| LNO₂ (10 and 12-nitrolinoleic acid) | 0.01% |
| OA-NO₂ (10-nitrooleic acid) | 0.01% |
| PEG-200 | q.s. to 100%\grave{ } |
| PEG-400 | 2.00% |
| Labrasol | 2.00% |
| Oleth-3 | 1.00% |
| Diglycerin | 1.00% |
| Oxynex AP | 0.01% |

### Example 2

A nitroalkene topical composition formulation is as follows.

| Component | % w/w |
|---|---|
| LNO₂ | 0.025% |
| PEG-200 | q.s. to 100%\grave{ } |
| PEG-400 | 5.00% |
| Propylene glycol | 1.00% |
| BV-OSC | 1.00% |
| VC-IP | 1.00% |
| Beta carotene | 0.25% |
| Diisopropyl adipate | 5.00% |
| Oxynex AP | 0.01% |

### Example 3

A nitroalkene topical composition formulation is as follows.

| Component | % w/w |
|---|---|
| LNO₂ | 0.02% |
| Diglycerin | q.s. to 100%\grave{ } |
| Phytantriol | 0.10% |
| Panthenol | 0.10% |
| Glycereth-4 | 10.00% |
| Ceraphyl ® 41 | 5.00% |
| Oleth-3 | 1.00% |
| Oxynex AP | 0.01% |

### Example 4

Two nitroalkene emulsion topical composition formulations are as follows.

| Component | % w/w | % w/w |
|---|---|---|
| PEG-400 | q.s. to 100% | q.s. to 100% |
| Ethoxylated glycerin | 15.00% | — |
| Glycerin | 5.00% | 5.00% |
| NaCL | 0.10% | 0.10% |
| BV-OSC | 1.00% | 1.00% |
| Mineral Oil | 25.00% | 25.00% |
| Dow Corning ® Fluid 244 | 2.00% | 2.00% |
| (methylsiloxane fluid) | | |
| Abil WE-09 | 5.00% | 5.00% |
| (Polyglyceryl-4 Isostearate and | | |
| Cetyl Dimethicone Copolyol and | | |
| Hexyl Laurate) | | |
| Cranberry seed oil | 1.00 | — |
| LNO₂ (or OANO₂) | 0.01% | 0.025% |
| Oxynex AP | 0.01% | 0.01% |

### Example 5

Two nitroalkene emulsion topical composition formulations are as follows.

| Component | % w/w | % w/w |
|---|---|---|
| PEG-400 | q.s. to 100% | q.s. to 100% |
| Glycerin | 5.00% | 5.00% |
| NaCL | 0.10% | 0.10% |
| LNO₂ (or OANO₂) | 0.01% | 0.01% |
| Dow Corning ® Fluid 245 | 15.00% | 15.00% |
| (cyclopentasiloxane fluid) | | |
| Dow Corning Fluid ® 3225 C | 9.00% | 9.00% |
| (silicone surfactant in dimethylsiloxane) | | |
| Tween 2 | 1.50 | 1.50 |
| (polysorbate 20) | | |
| Oxynex AP | 0.01% | 0.01% |

Therapeutic Uses of The Compositions

Generally in the practice of the methods of the invention, the topical composition is topically applied to the skin areas, such as that of the face, at predetermined intervals with gradual improvement in the skin areas expected with each successive application.

Topical compositions containing nitroalkene according to the present invention can be topically applied to and absorbed by the skin tissue. Nitroalkenes activate Nrf2, PPARγ, modify NF-kB gene expression or inhibit IFN-γ and TNF and human neutrophils and macrophage degranulation as well as cytokine release, thus preventing the cascade of reactions that lead to inflammation and degranulation.

While not wishing to be bound by any theory, it is possible that nitroalkenes react with cellular nucleophiles to postranslationally modify protein structure thus affecting XOR, GAPDH or any of the aforementioned inflammatory regulating compounds. Another possibility is that nitro compounds of the present invention work in a similar manner to the polyunsaturated nitro compounds of Ferrante. Moreover, nitroalkenes of the invention may enhance Nrf2 nuclear translocation, activate PPARγ, or modify the NF-kB subunit 65 that encodes proinflammatory cytokines.

Nitroalkenes undergo solvation in aqueous solutions and therefore tend to decay faster in phosphate buffer than in organic solvent. Release of .NO is also observed in aqueous environments. The release is independent of the presence of thiol adjuvants such as cysteines. The capacity to release .NO is has been related to the vasorelaxing properties of nitroalkenes observed in rat aortic ring, specifically AANO₂ and AA(OH)NO₂. The stability of nitro-fatty acids is better in hydrophobic environments. The release of .NO from LNO₂ is inhibited when inserted in phosphatidylcholine/cholesterol liposomes and it is considered stable in hydrophobic environments.

Methods and compositions of the present invention are expected to be particularly useful for treating skin tissue suffering from or damaged by inflammatory conditions. The methods and compositions are expected to be useful in prevention and treatment of the following conditions: rosacea, eczema, psoriasis, xerosis, dermatitis (both contact dermatitis and atopic dermatitis), seborrhea, thermal and radiation burns (including sunburn), acne, alopecia, aging-induced skin tissue degeneration, scars, and other types of skin inflammation.

Skin aging bears some similarities to chronic inflammatory conditions. Cell aging is due in part to free radical damage, which takes place mostly within the cell membrane. The cell membrane is most susceptible to attack by free radicals because of its dense molecular structure largely comprising lipids and lipoproteins, which are easily oxidized by reactive oxygen species. In skin, reactive oxygen species such as singlet oxygen, the superoxide anion, and hydroxyl radicals, as well as other free radicals, are generated in normal metabolism, as well as through ultraviolet sun exposure, other forms of radiation, other environmental factors such as pollution or exposure to chemicals in the home or workplace, and the like, active in the arachidonic acid cascade. As in inflammation, free radicals activate chemical mediators that increase phospholipidase A2 resulting in the release of arachidonic acid from the cell membrane which is then oxidized by lipooxygenase and cyclooxygenase enzymes which produce leukotrines and prostaglandins, stimulating the inflammation cascade. It is expected that the topical application of nitroalkene compounds according to the invention will be effective to protect collagen and elastin from degradation by matrix metallopritenases. After treatment for a period of time, it is expected that elasticity and a supple feeling will return to the skin, fine lines and wrinkles will be reduced, and skin coloring will even out. The present invention thus includes use of nitroalkene compositions to prevent and treats skin aging, as well as both preventing and treating skin damage.

Skin inflammation appears in many conditions of alopecia, inclusing male pattern baldness. I believe that the compositions and methods of the present invention will also be effective to prevent or treat alopecia by regular application of nitroalkene compositions.

Another expected use of the compositions of the present invention is in encouraging wound healing without scarring, and also, in remodeling scarred skin to a smoother, unscarred appearance. Scars result from wound healing, which occurs in three separate phases: inflammation, formation of granulation tissue, and matrix formation. (See Plast. Reconst. Surgery, 2008 122:1068-78; incorporated herein by reference). During the first phase, damage to endothelial cells, complement, and platelets at the wound site release chemotactic factors that result in the infusion of neutrophils, lymphocytes and macrophages, which aids in the removal of infection and foreign debris. As in all inflammatory processes, there is generation of free radicals, which damages cell membranes and results in formation of oxidized proteins and fats, and cross-linked new collagen, laying a scaffold for the next phase. At the end of the inflammatory phase, the granulation phase begins with an influx of fibroblasts and endothelial cells to the wound. Other key cells in this phase are macrophages and platelets. Macrophages induce the beginning of granulation by releasing platelet-derived growth factor (PDGF), tumor necrosis growth factor (TGF)-α, and an epidermal growth factor-like substance. Activated platelets release epidermal growth factor (EGF), PDGF, TGF-α, and TGF-α. Together these play roles in the re-epithelialization process wherein keratinocytes cells migrate in sheaths over a provisional matrix consisting primarily of fibrin, fibronectin, type V collagen, and tenascin, and produce their own fibronectin receptors. Once re-epithelilization has occurred, keratinocytes resume their normal differentiated form, and matrix formation begins. Matrix formation consists primarily of the construction of dermal matrix, which is regulated by fibroblasts. Chemotaxis of fibroblasts results in the production of abundant quantities of hyaluronate, fibronectin, and types I and III collagen. These components comprise the bulk of the provisional extracellular matrix in the early part of this wound repair phase. Hyaluronic acid (HA) creates an open-weave pattern in the collagen/fibronectin scaffold, facilitating fibroblast movement. HA production falls after about the fifth day of wound healing, and levels of chronroitin sulfate in dermatan sulfate increase. Fibronectin deposits in the collagen, and wound contraction begins. Biochemically during the contraction stage, hyaluronidase and proteinase are present, type I collagen synthesis is stimulated, and increased levels of chronroitin sulfate, dermatin sulfate and proteoglycans are observed; together these restructure the matrix. At the end of the healing process, the final scar shows collagen fibers mostly parallel to the epidermis.

Hypertrophic and keloid-type scars result in extension of scar tissue so that a bulky lesion results. A keloid is an exuberant scar that proliferates beyond the original wound. It should be noted that keloids only occur in humans, often causing burning, stinging and itching sensations as well as cosmetic embarrassment. The etiology of unsightly keloid formation is not known. However, in keloids, fibronectin formation continues for years, while fibronectin formation in normal scars disappears within a few days after wound closure. Keloid scars exhibit a high rate of collagen synthesis in comparison to normal scars, and a low proportion of cross-linked collagen. Hypertrophic scars sometimes are difficult to distinguish from keloid scars histologically and biochemically, but unlike keloids, hypertropic scars remain confined to the injury site and often mature and flatten out over time. Both types secrete larger amounts of collagen than normal scars, but typically the hypertrophic type exhibits declining collagen synthesis after about six months. However, hypertro-phic scars contain nearly twice as much glycosaminoglycan as normal scars, and this and enhanced synthetic and enzymatic activity result in significant alterations in the matrix which affects the mechanical properties of the scars, including decreased extensibility that makes them feel firm.

Atrophic scars are characterized by a thinning and diminished elasticity of the skin due to a loss of normal skin architecture. An example of an atrophic scar is striae distensae, also known as stretch marks. Striae commonly occur in postpartum women after childbirth and also during times of larger-than-average weight gain and also in association with steroids. Atrophic scars are sometimes also observed after trauma, infection and disease, and may show loss of surface markings and smoothness or dry, fine wrinkles over time.

Formation of scars, especially hypertrophic and keloid scars, is dependent on systemic growth factors such as interleukins and other cytokines, and their influence on fibronectin and collagen biossynthesis. Cytokines are released and are present in the wound healing process and, as mentioned above, are released in the inflammatory stage. Growth factors and other cytokines vary in the inflammatory stage and are released in amounts based, among other complex interactions, upon the redox state of the cells. The presence of free radicals in the inflammatory stage plays an important factor in wound healing. Factors that increase the presence of free radicals, such as infection, radiation, and continued trauma, may instigate hypertrophic and keloid scar formation. It is important to note that cytokines have been suggested to regulate nitric oxide synthetase, which controls the formation of nictric oxide, which plays an important role in signal transduction in the cells. It is also known that nitric oxide synthetase activity is aberrant in keloid scars when compared to normal tissue (Lim, T. C., et al., Plastic and Reconst. Surgery, 1996, 98: 911-912). Hypertrophic and keloid scars also show inflammatory activity that is not seen in mature scars.

The nitroalkene compositions and methods of the present invention are expected to be effective to reduce scarring during the process of wound healing and to remodel previously damaged or scarred skin. After treatment for a period of time, decreased inflammation, irritation, and erythema of the skin should occur, with a flattening of the scars and evening out of skin coloring.

Acne is the most common pustular condition of the skin, disfiguring afflicted persons with inflammatory and noninflammatory lesions (including pustules, papules and comedones) during the active phase, and with atrophic scars afterwards. It occurs most commonly in teenagers, but is not confined to adolescents. A significant number of persons continue to seek advice on treatment for acne after the teenage years (Collier et al., J. Am. Acad. Dermatology, 2008 58:56-59). Although acne is generally considered to be self-limiting, its social effects can be substantial, and it may have its most severe effects on the psyche (Am. J. Clinical Dermatology, 2008 9(5):279-284). In about 60% of teenagers, disease severity and embarrassment are sufficient for them to self-medicate with proprietary preparations and/or seek medical advice.

Acne is a multifactorial disease affecting the pilosebaceous units of the skin. Each unit consists of a large, multilobed sebaceous gland, a rudimentary hair and a wide follicular canal lined with stratefied squamous epithelium. They are found over most of the body surface but are largest and most numerous on the face, chest, and upper back. Normally, desquamated follicular cells are carried to the surface by the flow of sebum. Under the abnormal circumstances of acne vulgaris, an abnormal desquamination process provokes increased sloughing of the epithelium, which becomes more cohesive because of defective keratinization. This process causes blockage of the follicular orifice with accumulation of dead cells. Androgen stimulates the undifferentiated hormonally responsive cells making up the outer layer of the sebaceous gland lobule to divide and differentiate. Sebum production favors proliferation of the anaerobe Propionibacterium acnes, which is a normal commensal to the pilosebaceous unit, which can elicit hypersensitivity responses in acne.

The aims of treating acne are to minimize the number and severity of lesions, prevent scarring, limit disease duration, and reduce the social and psychological stress that affects many patients, particularly teenagers. Conventional treatment is directed at correcting the three major factors that seem to cause acne: (1) androgenic stimulation of the sebaceous glands and increased sebum production; (2) abnormal keratinization and impaction in the pilosebaceous canal causing obstruction to sebum flow; and (3) proliferation of P. acnes. Thus, topical agents that remove comedones, such as topical retinoids are particularly effective because they normalize desquamination within the follicular orifice, which allows the sebum to flow freely onto the surface of the skin; adalpalene, tretinoin, and tazarotene have been shown to have efficacy in treating mild to moderate acne, but all three have reported to have skin-irritating side effects including erythema, pruritis, burning/stinging, and scaling/flaking (Physicians' Desk Reference®, 56th ed. 2002, p. 2523, hereinafter referred to as “PDR”). The side effects of retinoid use are so extreme that many individuals cannot tolerate topical application of these agents at all.

The nitroalkene compositions and methods of the present invention are expected to be effective to prevent and to treat acne. After treatment for a period of time, decreased inflammation, irritation and erythema of the skin. This should result in an elimination of acne and repair microscarring of the dermis from prior acne lesions.

Psoriasis is another inflammatory skin disease that occurs when faulty signals in the immune system cause keratinocyte skin cells to regenerate too quickly, on the order of every three to four days instead of the usual 30-day cycle. Extra skin cells build up on the skin's surface, forming red, flaky, scaly lesions that can itch, crack, bleed and be extremely painful. Psoriasis generally involves the joints, limbs and scalp but it can appear anywhere on the body, covering some people from head to toe. More than 5 million Americans have been diagnosed with psoriasis and/or psoriatic arthritis, a degenerative disease of the joints and connective tissues associated with psoriasis. Psoriasis typically first strikes people between the ages of 15 and 35, but can affect anyone at any age, including children.

Psoriasis is characterized by erythematous eruptions, often in papules or plaques, and usually having a white, silvery scale. Numerous studies have identified tumor necrosis factor α(TNF-α) as a particularly relevant cytokine regulating this complex inflammatory cascade. Its key role is underlined by the therapeutic efficacy of compounds that interfere with TNF-α functions. It is thought that neutrophils, another leukocyte population abundantly present in psoriatic infiltrates, are recruited by the neutrophil-attracting chemokine interleukin-8 (CXCL8). However, this pathway is probably not the exclusive means of neutrophil recruitment.

The nitroalkene compositions and methods of the present invention are expected to be effective to prevent and to treat psoriasis.

The present invention thus prevents skin aging and treats skin aging, as well as both preventing and treating skin damage including inflammation, scarring and erythema.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method for the prevention of skin aging comprising: topically applying a composition containing an effective amount of a nitroalkene in a dermatologically acceptable non-polar carrier to skin tissue.

2. The method of claim 1, wherein the nitroalkene is nitro-linoleic acid, nitro-oleic acid, nitrated arachidonic acid, or nitrated cholesteryl lineolate.

3. The method of claim 1 wherein the nitroalkene is present in a weight percentage which is within one of the following ranges:
0.01%-0.025%;
0.025%-0.05%;
0.05%-0.10%;
0.10%-0.50%;
0.50%-1.0%;
0.025%-0.50%;
0.025%-1.0%;
1.0%-2.0%;
2.0%-5.0%;
5.0%-10.00%;
1.0%-5.0%;
1.0%-10.0%;
10.0%-20.0%;
20.0%-30.0%;
30.0%-40.0%;
40.0%-50.0%;
50.0%-60.0%;
60.0%-70.0%;
70.0%-80.0%;
80.0%-90.0%;
90.0%-98.0%;
10.0%-30.0%;
20.0%-40.0%;
30.0%-60.0%;
40.0%-70.0%;
50.0%-80.0%;
10.0%-50.0%;
10.0%-98.0%;
50.0%-70.0%;
50.0%-98.0%; or
70.0%-98.0%.

4. The method of claim 1, wherein the non-polar carrier comprises one or more of: polyethylene glycols, glycerides, glycerin; polypropylene glycol; PVM/MA Decadiene Crosspolymer; hydrogenated polyisobutane/polyethane; isododecane; tetrahexyldecyl ascorbate; Vitamine E; beta carotene; disopropyl adipate; 2-ethylhexyl pentate; oleth-3; propanoic acid 2-hydroxy-dodecyl ester; propanoic acid, 2-hydroxy-, C12-15-alkyl esters; glycereth-4; glycereth-7; diglycerin; panthenol; and phytantriaol.

5. The method of claim 4, wherein the non-polar carrier comprises one or more polyethylene glycols.

6. The method of claim 1, wherein said composition further comprises one or more additional ingredients selected from the group consisting of: ascorbyl palmitate, lipoic acid, and tocotrienols and tocotrienol derivatives and vitamin E compositions enriched with tocotrienol or tocotrienol derivatives.

7. A method for the treatment of skin aging comprising: topically applying a composition containing an effective amount of a nitroalkene in a dermatologically acceptable non-polar carrier to skin tissue showing conditions of skin aging.

8. The method of claim 7, wherein the nitroalkene is nitro-linoleic acid, nitro-oleic acid, nitrated arachidonic acid, or nitrated cholesteryl lineolate.

9. The method of claim 7 wherein the nitroalkene is present in a weight percentage which is within one of the following ranges:
0.01%-0.025%;
0.025%-0.05%;
0.05%-0.10%;
0.10%-0.50%;
0.50%-1.0%;
0.025%-0.50%;
0.025%-1.0%;
1.0%-2.0%;
2.0%-5.0%;
5.0%-10.00%;
1.0%-5.0%;
1.0%-10.0%;
10.0%-20.0%;
20.0%-30.0%;
30.0%-40.0%;
40.0%-50.0%;
50.0%-60.0%;
60.0%-70.0%;
70.0%-80.0%;
80.0%-90.0%;
90.0%-98.0%;
10.0%-30.0%;
20.0%-40.0%;
30.0%-60.0%;
40.0%-70.0%;
50.0%-80.0%;
10.0%-50.0%;
10.0%-98.0%;
50.0%-70.0%;
50.0%-98.0%; or
70.0%-98.0%.

10. The method of claim 7, wherein the non-polar carrier comprises one or more of: polyethylene glycols, glycerides, glycerin; polypropylene glycol; PVM/MA Decadiene Crosspolymer; hydrogenated polyisobutane/polyethane; isododecane; tetrahexyldecyl ascorbate; Vitamine E; beta carotene; disopropyl adipate; 2-ethylhexyl pentate; oleth-3; propanoic acid 2-hydroxy-dodecyl ester; propanoic acid, 2-hydroxy-, C12-15-alkyl esters; glycereth-4; glycereth-7; diglycerin; panthenol; and phytantriaol.

11. The method of claim 10, wherein the non-polar carrier comprises one or more polyethylene glycols.

12. The method of claim 7, wherein said composition further comprises one or more additional ingredients selected from the group consisting of: ascorbyl palmitate, lipoic acid, and tocotrienols and tocotrienol derivatives and vitamin E compositions enriched with tocotrienol or tocotrienol derivatives.

13. A method for preventing skin aging mediated by free radicals, said method comprising topically applying to skin areas to be treated a composition containing at least one nitroalkane compound in an amount sufficient to provide free radical scavenging in the skin.

14. The method of claim 13, wherein the nitroalkene is nitro-linoleic acid or nitro-oleic acid.

15. The method of claim 14, wherein the composition comprises one or more of: polyethylene glycols, glycerides, glycerin; polypropylene glycol; PVM/MA Decadiene Crosspolymer; hydrogenated polyisobutane/polyethane; isododecane; tetrahexyldecyl ascorbate; Vitamine E; beta carotene; disopropyl adipate; 2-ethylhexyl pentate; oleth-3; propanoic acid 2-hydroxy-dodecyl ester; propanoic acid, 2-hydroxy-, C12-15-alkyl esters; glycereth-4; glycereth-7;
diglycerin; panthenol; and phytantriaol.

16. The method of claim 15, wherein the composition comprises one or more polyethylene glycols.

17. The method of claim 16, wherein said composition further comprises one or more additional ingredients selected from the group consisting of: fatty acid ester of ascorbic acid, lipoic acid, and tocotrienols and tocotrienol derivatives and vitamin E compositions enriched with tocotrienol or tocotrienol derivatives.

18. A method according to claim 17 wherein the fat-soluble fatty acid ester of ascorbic acid is selected from the group consisting of ascorbyl palmitate, ascorbyl laurate, ascorbyl myristate, ascorbyl stearate, and mixtures thereof.

19. A method according to claim 18 wherein the fat-soluble fatty acid ester of ascorbic acid is ascorbyl palmitate.

20. A method for treating skin aging, said method comprising topically applying to skin areas to be treated a composition containing at least one nitroalkane compound in an amount sufficient to provide protein production in the skin areas and/or remodeling of skin cell proteins.

21. A method according to claim 20 in which the composition further comprises a dermatologically acceptable carrier.

22. The method of claim 21, wherein the nitroalkene is nitro-linoleic acid, nitro-oleic acid, nitrated arachidonic acid, or nitrated cholesteryl lineolate.

23. The method of claim 20, wherein said composition further comprises a fatty acid ester of ascorbic acid.

24. A method according to claim 23 wherein the fat-soluble fatty acid ester of ascorbic acid is selected from the group consisting of ascorbyl palmitate, ascorbyl laurate, ascorbyl myristate, ascorbyl stearate, and mixtures thereof.

25. A method for the prevention and treatment of skin aging comprising topically applying to skin areas to be treated a composition containing at least one nitroalkane compound in an amount sufficient to provide free radical scavenging and protein production in the skin areas and/or remodeling of skin cell proteins.

26. A method according to claim 25 in which the composition further comprises a dermatologically acceptable carrier.

27. The method of claim 25, wherein the nitroalkene is nitro-linoleic acid, nitro-oleic acid, nitrated arachidonic acid, or nitrated cholesteryl lineolate.

28. The method of claim 25, wherein said composition further comprises a fatty acid ester of ascorbic acid.

29. A method according to claim 28 wherein the fat-soluble fatty acid ester of ascorbic acid is selected from the group consisting of ascorbyl palmitate, ascorbyl laurate, ascorbyl myristate, ascorbyl stearate, and mixtures thereof.
